(19) United States
(12) Patent Application Publication
(10) Pub. No.: US 2021/0052413 A1
(43) Pub. Date: Feb. 25, 2021

(54) MOBILE JOINT RESTRAINT

(71) Applicants: Damian Scavo, Menlo Park, CA (US); Karolina Kaminska, Menlo Park, CA (US)

(72) Inventors: Damian Scavo, Menlo Park, CA (US); Karolina Kaminska, Menlo Park, CA (US)

(21) Appl. No.: 16/997,594

(22) Filed: Aug. 19, 2020

Related U.S. Application Data
(60) Provisional application No. 62/888,993, filed on Aug. 19, 2019.

(51) Int. Cl. A61F 5/37; B33Y 10/00; B33Y 80/00

(52) U.S. Cl. CPC A61F 5/373; B33Y 80/00; B33Y 10/00

(57) ABSTRACT

A mobile joint restraint for treating a medical condition for which the mobile joint restraint is required is provided. The mobile joint restraint may include a first part comprising a pair of cut-out pieces, a pair of holes with a strap configured to secure the first part to a limb, and a pair of holes at the ends of arms of the first part configured to attach to a second part. The second part may comprise a pair of restricting protrusions configured to sit within the cut-out piece, a pair of holes with a strap configured to secure the second part to a limb, and a pair of flanges at the ends of arms of the second part configured to couple with the pair of holes at the ends of the arms of the first part.

### CROSS-REFERENCE TO RELATED APPLICATION(S)

This application claims the benefit of U.S. Patent Application No. 62/888,993, filed Aug. 19, 2019, the disclosure of which is hereby incorporated by reference in its entirety.

### BACKGROUND

### Field

The present disclosure relates to a mobile arm restraint, and more specifically, to systems and methods for restricting movement of a ligament while providing adequate flexibility and comfort.

### Related Art

Children who have been treated for medical conditions such as cleft palate may be required to keep their arms in a structurally static brace, in order to prevent the child from scratching or disturbing their surgical site, for example. In related art implementations, these braces may be rigid, immobilizing the portions of the child's wrist and elbow, to restrict movement. However, these rigid braces may not allow for comfort or flexibility, which may contribute to muscular atrophy and prolonged injury.

Alternatively, if a child has a small sprain or other injury which may require some immobilization, elastic bandages and sports tape may be used to provide support in some related art implementations. However, these solutions may not be the most effective for a child, because of the ease of removal, the lack of durability, and the lack of adequate support for an active child. For example, a child, in most circumstances, is less patient than an adult, and thus may remove the tape themselves, if they experience any discomfort. Additionally, children tend to be more active day to day, so an elastic bandage or a sports tape may provide a long-term solution for healing a child's injury.

### SUMMARY OF THE DISCLOSURE

Aspects of the present disclosure include a mobile restraint for treating a medical condition for which the mobile restraint is required. The mobile restraint may include a first part comprising a pair of cut-out pieces, a pair of holes with a strap configured to secure the first part to a limb, and a pair of holes at the ends of arms of the first part configured to attach to a second part.

The second part may comprise a pair of restricting protrusions configured to sit within the cut-out piece, a pair of holes with a strap configured to secure the second part to a limb, and a pair of flanges at the ends of arms of the second part configured to couple with the pair of holes at the ends of the arms of the first part.

The first part and the second part may each comprise a plurality of holes at a curved surface opposite the arms of the first part and the second part.

The mobile joint restraint may be made of a versatile plastic.

The pair of cut-out pieces may include tabs for adjusting the degree of mobility.

Further, the cut-out piece may allow for multiple degrees of mobility.

Additional aspects of the present disclosure include a method for treating a medical condition for which a mobile restraint is required. The method may comprise providing a first part at an upper part of a limb, the first part comprising a pair of cut-out pieces, a pair of holes with a strap configured to secure the first part to the limb, and a pair of holes at the ends of arms of the first part configured to attach to a second part.

The method may further comprise providing the second part at a lower part of the limb, the second part comprising a pair of restricting protrusions configured to sit within the cut-out piece, a pair of holes with a strap configured to secure the second part to a limb, and a pair of flanges at the ends of arms of the second part configured to couple with the pair of holes at the ends of the arms of the first part.

Additionally, the method may further comprise coupling the first part to the second part by providing the pair of flanges of the second part through the pair of holes at the ends of arms of the first part, and securing the straps of the first part and the second part to the upper part and the lower part of the limb.

Further aspects of the present disclosure include a method of manufacturing a mobile restraint for treatment of a medical condition. The method may comprise: obtaining measurements of a limb and other information, determining an allowable range of motion, inputting the data, printing the mobile restraint with a 3-D printer, clasping a first part to a second part, wherein the first part has a pair of holes and the second part has a pair of flanges, wherein the pair of flanges are passed through the pair of holes, and securing the mobile restraint with a fastener located at each of the first part and the second part.

### BRIEF DESCRIPTION OF THE DRAWINGS

FIG. 1 illustrates a top view of a mobile joint restraint, according to some example implementations.

FIG. 2 illustrates a bottom view of the mobile joint restraint, according to some example implementations.

FIG. 3 illustrates a peripheral view of a first part of the mobile joint restraint, according to some example implementations.

FIG. 4 illustrates a peripheral view of another example of a first part of the mobile joint restraint, according to some example implementations.

FIG. 5 illustrates a top view of a first part of the mobile joint restraint, according to some example implementations.

FIG. 6 illustrates a bottom view of a first part of the mobile joint restraint, according to some example implementations.

FIG. 7 illustrates a peripheral view of a second part of the mobile joint restraint, according to some example implementations.

FIG. 8 illustrates a top view of a second part of the mobile joint restraint, according to some example implementations.

FIG. 9 illustrates a bottom view of a second part of the mobile joint restraint, according to some example implementations.

FIGS. 10A and 10B illustrate a side view and a top view, respectively, of a first part of the mobile joint restraint, according to some example implementations.

FIGS. 11A and 11B illustrate a side and a top view, respectively, of a second part of the mobile joint restraint, according to some example implementations.

FIG. 12 illustrates a method for creating a mobile joint restraint, according to some example implementations.

### DETAILED DESCRIPTION

The following detailed description provides further details of the figures and example implementations of the present application. Reference numerals and descriptions of redundant elements between figures are omitted for clarity. Terms used throughout the description are provided as examples and are not intended to be limiting.

Thus, aspects of the present disclosure aim to provide a restraint with flexibility, directed to providing the child with comfort while using the restraint. While the present disclosure may describe example implementations of to an arm restraint at the elbow, example implementations may include restraints for the elbows, wrists, knees, and ankles, as well as any other joint area which may require support with some flexibility.

For example, a child who plays a sport may receive a sprained ankle. While a plaster cast or a walking cast may be used, the child's ankle may still be highly immobilized, increasing the risk for atrophy of underlying muscles which help to stabilize the joint. Thus, by using a mobile restraint to heal the sprained ankle, the child may heal more quickly and effectively.

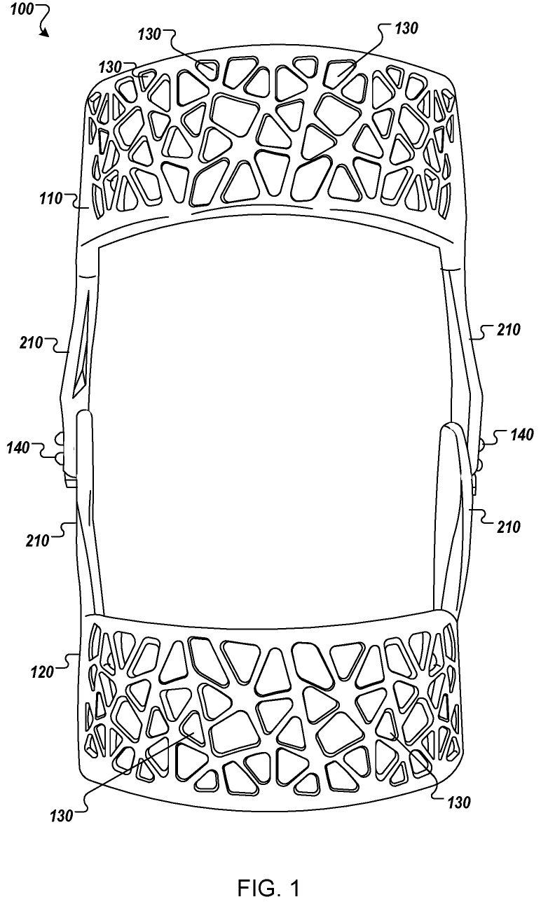

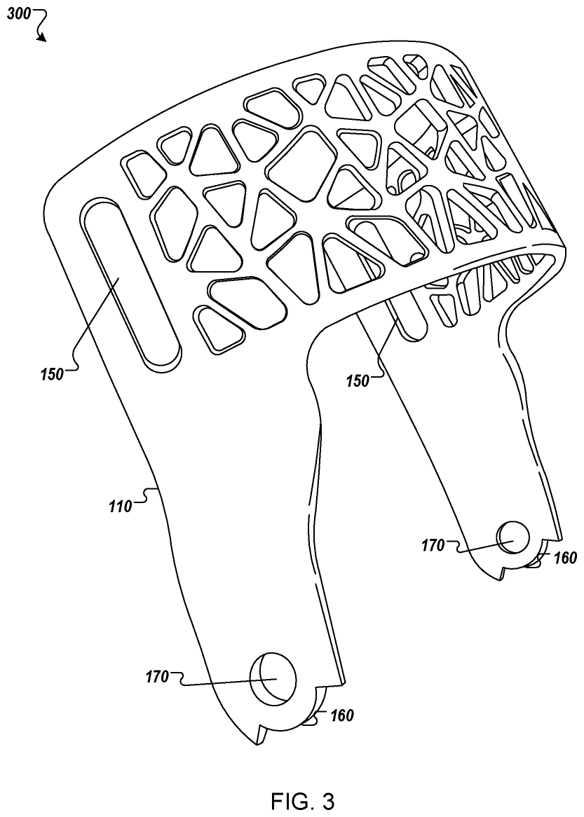

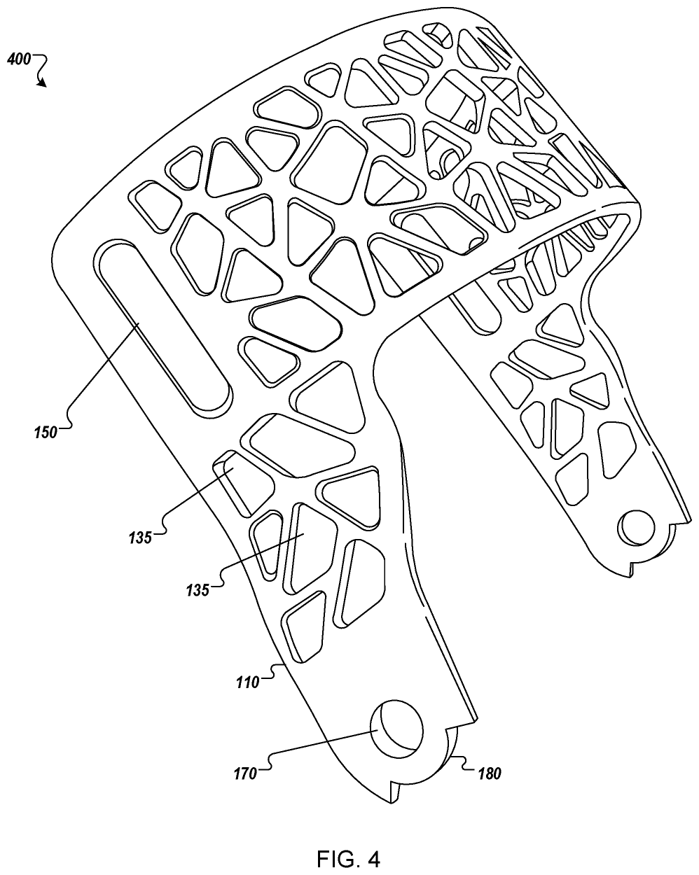

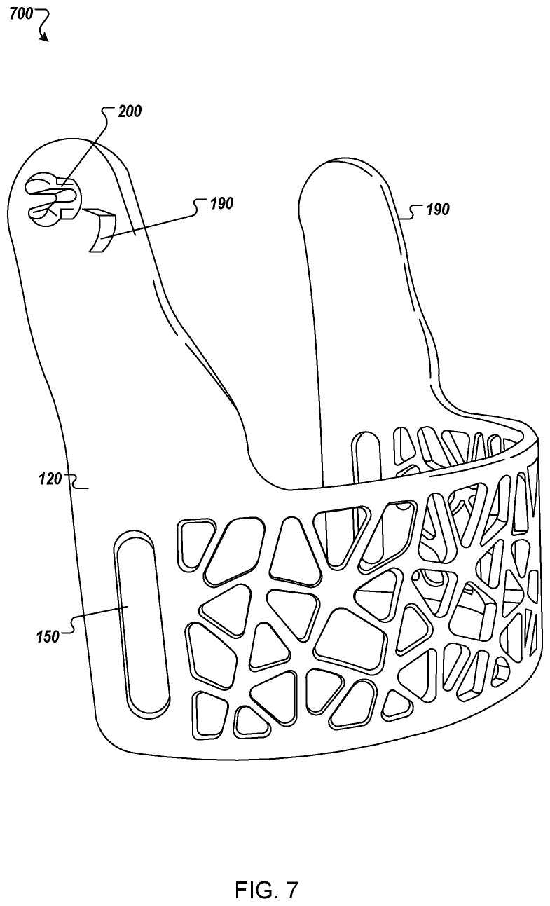

As shown in FIG. 1, the mobile joint restraint 100 may comprise a first part 110 and a second part 120. The first part 110 may have a cut-out piece 160, 180 (shown in FIGS. 3 and 4) and a hole 170 (shown in FIG. 3) on each arm 210, described in more detail below with respect to the other figures. The second part 120 may have a restricting protrusion 190 (shown in FIG. 7) and a flange 200 (shown in FIG. 7), also described in more detail below with respect to the other figures.

The first part 110 and the second part 120 may each have a curved face comprising a plurality of holes 130. Further, the first part 110 and the second part 120 may be joined together at a connecting portion 140. Connecting portion 140 may be provided at each of the arms 210 of the first part 110 and the second part 120.

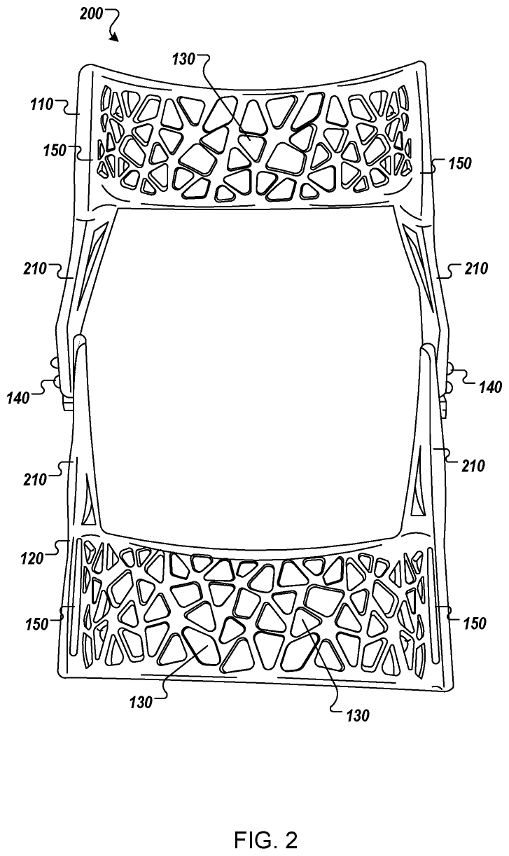

FIG. 2 shows an example of the mobile joint restraint 100 from an underside view 200. This is the side that may contact the skin of a user. As shown in FIG. 2, each of the arms 210 of the first part 110 and the second part 120 may have a securing hole 150 for securing the mobile joint restraint 100 to the user. For example, an elastic strap, a hook and loop strap (e.g., VELCRO), or any other type of fastening strap that might be apparent to a person of ordinary skill in the art may be passed through securing hole 150 for attaching the mobile joint restraint 100 to the user. It is preferable to use elastic, to provide comfort to the user, while fastening the elastic with a portion of a hook and loop strap. If a static strap were to be used, there may not be sufficient flexibility to compensate for the flexing and relaxing of a muscle.

Alternatively, snaps, hooks, or any other fasteners which may be apparent to one having ordinary skill in the art may be used, to secure the mobile joint restraint 100 to the user.

FIG. 3 illustrates an example top view 300 of the first part 110. Hole 170 may be provided to help fasten the second part 120 to the first part 110 at the connecting portion 140 of FIG. 1. Further, cut-out piece 160 may be provided at the ends of the arms of the first part 110, in order to restrict movement. For example, the degree of mobility and movement may be set depending on the size of the cut-out piece 160, described in further detail with respect to FIG. 4.

FIG. 4 illustrates another example of a top view 400 of the first part 110. In FIG. 4, cut-out piece 180 has a smaller opening than cut-out piece 160 of FIG. 3. This feature in FIG. 4 allows for more restriction of movement than the feature in FIG. 3. The mechanics of how this cut-out piece works will be described with respect to FIG. 7 below.

Various sizes for the cut-out piece 160 may be used. For example, a movement size of 30° may correspond to a particular size of a cut-out piece 160.

Additionally, a plurality of tabs may be provided at the ends of each of the cut-out pieces. These tabs may be removed at various times, in order to allow the range of mobility to change depending on where the user is in their recovery. For example, the mobile joint restraint may be initially provided with multiple tabs when the user requires a stricter range of motion. Later, one or more tabs may be removed by a doctor, practitioner, or user, in order to allow for a broader range of motion, as the user's condition is improving.

These tabs may assist in a user's rehabilitation after an injury, for example. By not totally restricting movement, as has been done in prior implementations, a user may have a small range of motion so as to prevent muscular atrophy. The user's joint is still restricted in motion, so that further injury is prevented, but rehabilitation may start earlier by use of the tabs.

These tabs may be torn off one by one and disposed of, or they may be secured back in place by any fastening means that might be apparent to a person of ordinary skill in the art.

FIG. 4 also illustrates the first part 110 having a plurality of holes 135 on the arms, and not just the curved part as shown in FIG. 3. The plurality of holes 130 on the curved part and the plurality of holes 135 on the arms may allow for more comfort to the user. For example, the plurality of holes 135 may allow for a user to scratch an itch when desired, and may prevent the restraint from sticking to the arm. In previous implementations, a user may have perspiration issues under the restraint because of the lack of breathability of the material. However, by providing the plurality of holes 130 and 135, the user may scratch an itch and may not need to worry about skin infections or rashes caused by the restraint agitating the surrounding skin.

Additionally, the plurality of holes 130 and 135 may vary in size, depending on the location of the restraint. For example, the holes may be larger for a larger area such as a knee area, to provide for better comfort and flexibility.

Because of the plastic being used and because of the plurality of holes 130 and 135, a user does not have to constantly clean the restraint, because the plurality of holes 130 and 135 allow for the restraint to breathe openly. Additionally, a user may wear the restraint on top of clothing if desired, because the restraint is sufficiently flexible to enable such wear.

Further, while FIG. 4 includes a plurality of holes 135 on the arms, it is preferable to have the arms be solid, as shown in FIG. 3, to provide more durability and reinforcement.

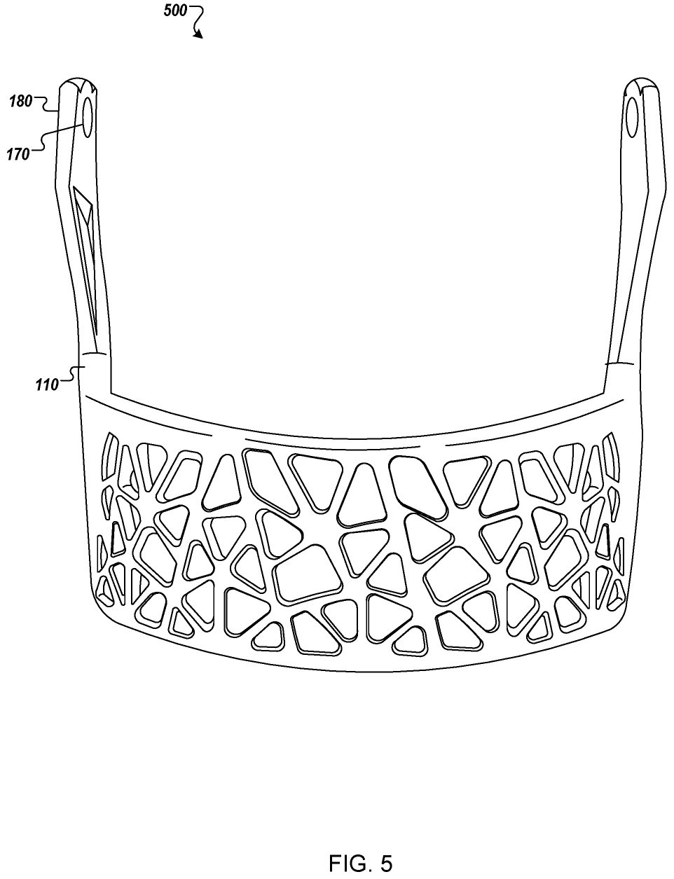

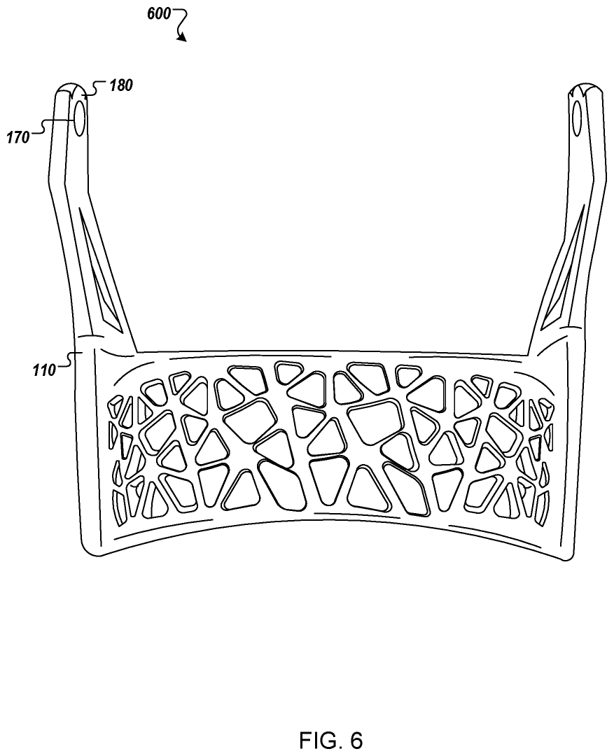

FIG. 5 illustrates an example top view 500 of the first part 110, and FIG. 6 shows an example bottom view 600 of the first part 110. In these views, the curves of the arms are shown. These curves enable a user to have comfort when flexing or relaxing a muscle group.

FIG. 7 illustrates an example view 700 of the second part 120. The second part 120 may have a restricting protrusion 190 and a flange 205 on each of the two arms of the second part 120. The restricting protrusion 190 may be inserted into the cut-out piece of the first part 110, in order to restrict the degree of movement. The restricting protrusion 190 may be of a fixed size, proportional to the size of the cut-out piece of the first part 110.

Further, the flange 205 may be fit through the holes 170 of the first part, in order to form the connecting portion 140. This flange may be any type of clasp or securing feature which allows for the first part 110 and the second part 120 to move.

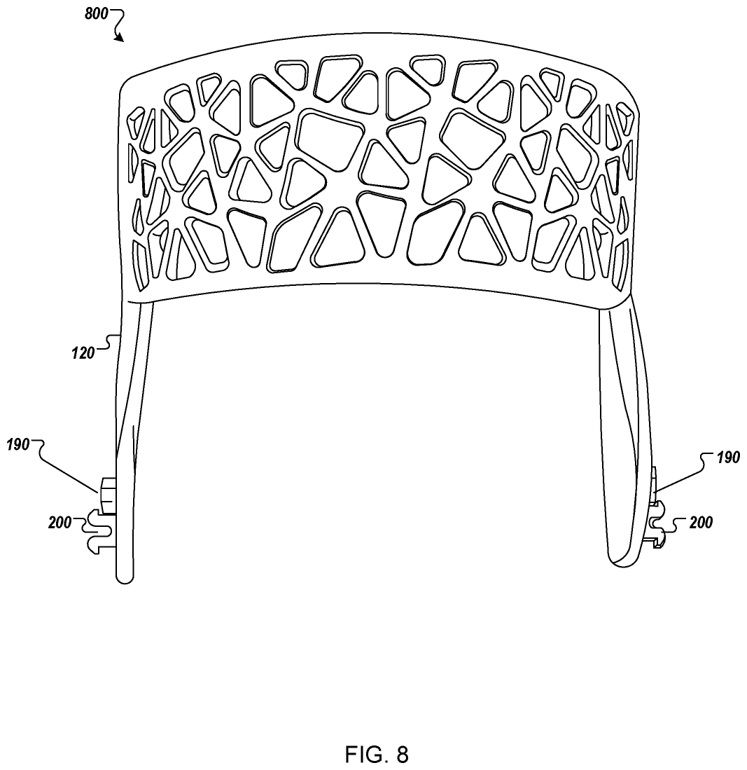

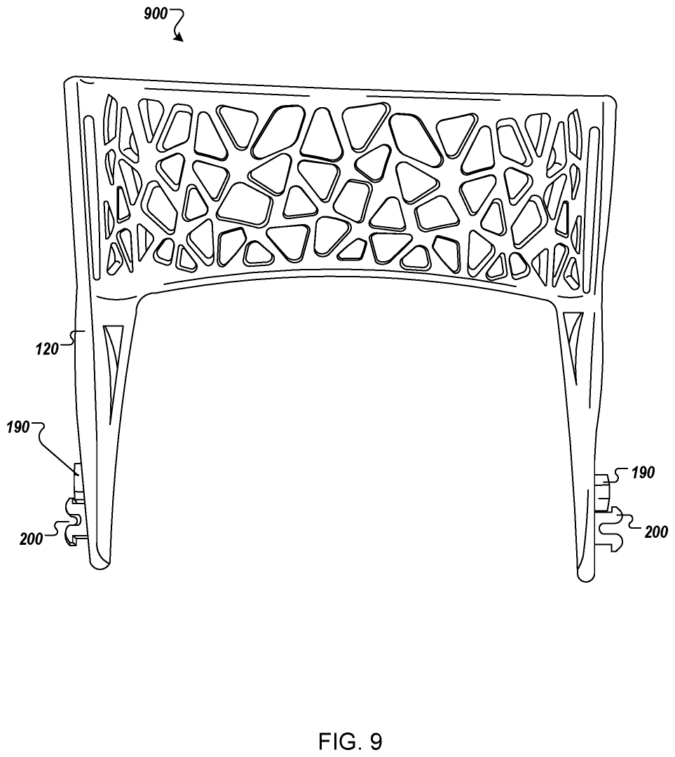

FIG. 8 illustrates an example top view 800 of the second part 120, and FIG. 9 shows an example bottom view 900 of the second part 120. In these views, the curves of the arms are shown. These curves enable a user to have comfort when flexing or relaxing a muscle group.

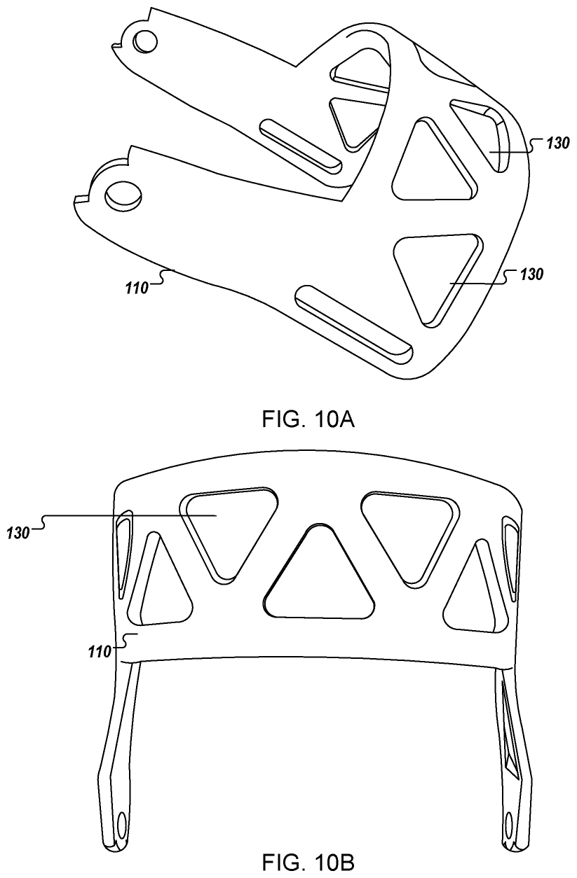

FIGS. 10A and 10B illustrate an example side view and top view of the first part 110, where a fewer number of the plurality of holes 130 are provided. While providing this plurality of holes may provide comfort to a user by allowing for flexibility, breathability, and ease of access to an underlying area, these plurality of holes may be configured in the manner shown in FIGS. 10A and 10B to provide more structural support for the affected area.

For example, if a user requires more restriction of movement and less flexibility, the mobile joint restraint 100 may be configured to provide fewer holes 130.

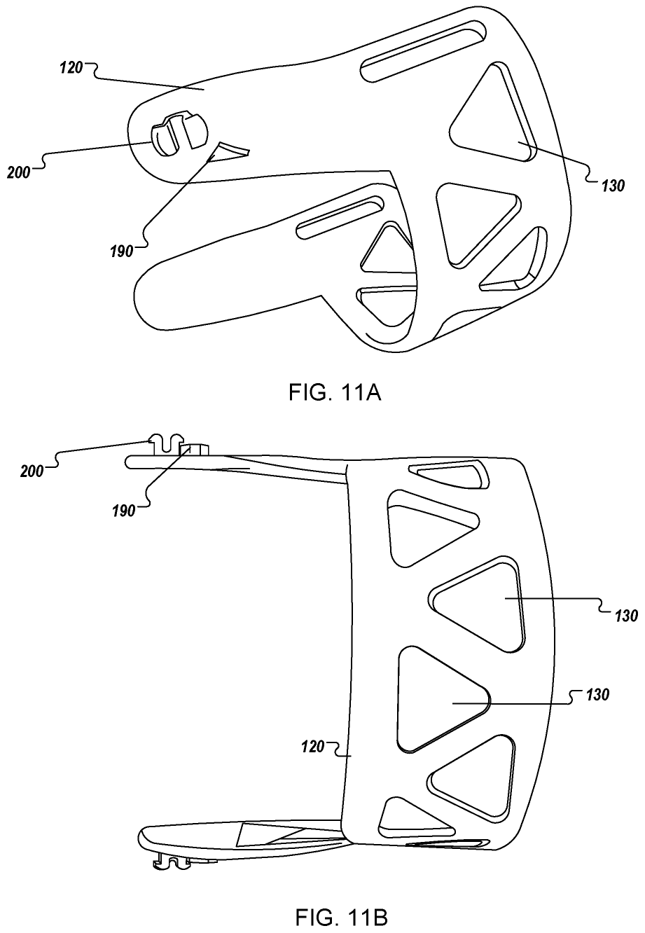

FIGS. 11A and 11B illustrate an example side view and top view of the second part 120, where a fewer number of the plurality of holes 130 are provided. Similarly, this plurality of holes 130 may be provided such that more or fewer holes 130 are printed, depending on the level of structural support required for proper treatment.

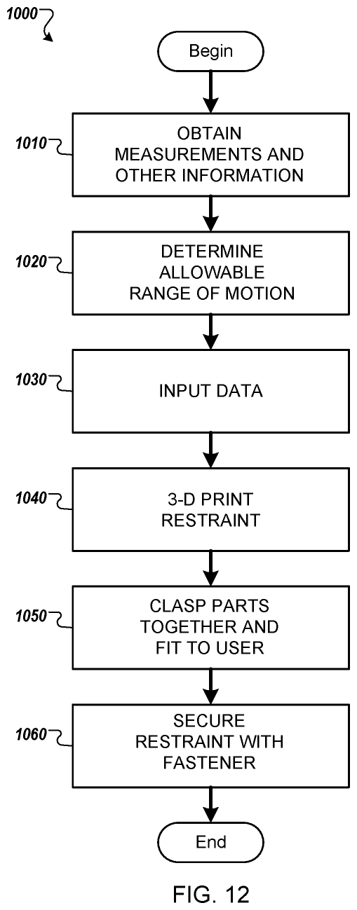

FIG. 12 illustrates an example method 1000 of making a mobile joint restraint such as mobile joint restraint 100. At step 1010, a doctor, user, or other technician may obtain measurements of the area to be treated. Other information, including age, mobility, type of injury, etc. may also be collected. Alternatively and/or additionally, a photo of the affected area may be taken and input into an application which can automatically determine the recommended size. By allowing the application to automatically determine a recommended size, a doctor may not be required for treatment, or a doctor may not need to be physically present for treatment.

Then, at step 1020, a doctor, user, or other technician may determine an allowable range of motion. For example, if a user should be able to move their arms but just not be able to reach their face, then one or more degrees of freedom to enable this range of motion may be chosen. Conversely, if a user should be nearly fully restricted, one or more degrees of freedom to enable this range of motion may be chosen.

At step 1030, this data may be input into an application or specification table. With this data, a 3-D printer may print the mobile joint restraint at step 1040. Various types of materials may be used to print the mobile joint restraint. For example, multi jet fusion plastic, versatile plastic, or fine detail plastic may be used. Alternatively, any material which may flex, return to its original un-flexed position without use of additional pressure, and is durable may be used. The material should ideally be lightweight to provide for comfort.

Further, the 3-D printer may print the mobile joint restraint in two parts, including a first part and a second part, such as the first part 110 and the second part 120.

Once the first part and the second part are printed, the two pieces may be connected at a connecting portion, such as connecting portion 140 at step 1050. To do this, a flange such as flange 205 of the second part 120 may be inserted into a hole such as hole 170 of the first part 110. Once inserted, a restrictive protrusion such as restrictive protrusion 190 may line up with the cut-out piece of the first part.

Once the pieces are clasped together and fit to the user at step 1050, the mobile joint restraint may be secured to the treatment area with a strap or other fastener at step 1060, where the strap may be inserted through securing holes, such as securing holes 150 of each of the arms of the first part and the second part. Alternatively, snaps or any other fastener which may be known to one having ordinary skill in the art may be used to connect the two ends.

Although a few example implementations have been shown and described, these example implementations are provided to convey the subject matter described herein to people who are familiar with this field. It should be understood that the subject matter described herein may be implemented in various forms without being limited to the described example implementations. The subject matter described herein can be practiced without those specifically defined or described matters or with other or different elements or matters not described. It will be appreciated by those familiar with this field that changes may be made in these example implementations without departing from the subject matter described herein as defined in the appended claims and their equivalents.

What is claimed is:

1. A mobile joint restraint for treating a medical condition for which the mobile joint restraint is required, the mobile restraint comprising:
a first part comprising a pair of cut-out pieces, a pair of holes with a strap configured to secure the first part to a limb, and a pair of holes at the ends of arms of the first part configured to attach to a second part; and
the second part comprising a pair of restricting protrusions configured to sit within the cut-out piece, a pair of holes with a strap configured to secure the second part to a limb, and a pair of flanges at the ends of arms of the second part configured to couple with the pair of holes at the ends of the arms of the first part.

2. The mobile joint restraint of claim 1, wherein the first part and the second part each comprise a plurality of holes at a curved surface opposite the arms of the first part and the second part.

3. The mobile joint restraint of claim 1, wherein the mobile joint restraint is made of a versatile plastic.

4. The mobile joint restraint of claim 1, wherein the pair of cut-out pieces include tabs for adjusting the degree of mobility.

5. A method of treating a medical condition for which a mobile restraint is required, the method comprising:
providing a first part at an upper part of a limb, the first part comprising a pair of cut-out pieces, a pair of holes with a strap configured to secure the first part to the limb, and a pair of holes at the ends of arms of the first part configured to attach to a second part;
providing the second part at a lower part of the limb, the second part comprising a pair of restricting protrusions configured to sit within the cut-out piece, a pair of holes with a strap configured to secure the second part to a limb, and a pair of flanges at the ends of arms of the second part configured to couple with the pair of holes at the ends of the arms of the first part;
coupling the first part to the second part by providing the pair of flanges of the second part through the pair of holes at the ends of arms of the first part; and
securing the straps of the first part and the second part to the upper part and the lower part of the limb.

6. The method of claim 5, wherein the first part and the second part each comprise a plurality of holes at a curved surface opposite the arms of the first part and the second part.

7. The method of claim 6, wherein the mobile joint restraint is made of a versatile plastic.

8. The method of claim 6, wherein the pair of cut-out pieces include tabs for adjusting the degree of mobility.

9. A method of manufacturing a mobile restraint for treatment of a medical condition, the method comprising:
obtaining measurements of a limb and other information;
determining an allowable range of motion;
inputting the data;
printing the mobile restraint with a 3-D printer;
clasping a first part to a second part, wherein the first part has a pair of holes and the second part has a pair of flanges, wherein the pair of flanges are passed through the pair of holes; and
securing the mobile restraint with a fastener located at each of the first part and the second part.
